(19) United States
(12) Patent Application Publication
(10) Pub. No.: US 2016/0323817 A1
(43) Pub. Date: Nov. 3, 2016

(54) SOIL MONITOR AND SYSTEM THEREFOR

(71) Applicant: Peyman Moeini, Toronto (CA)

(72) Inventor: Peyman Moeini, Toronto (CA)

(21) Appl. No.: 15/143,417

(22) Filed: Apr. 29, 2016

Related U.S. Application Data
(60) Provisional application No. 62/155,034, filed on Apr. 30, 2015.

(51) Int. Cl. H04W 52/02; G01N 33/24

(52) U.S. Cl. CPC H04W 52/0203; G01N 33/24

(57) ABSTRACT

In an aspect of the invention there is provided a module for use with a signal representative of time of activity instructions. The module comprises a power source in the form of one or more batteries, one or more sensors for receiving information, a transceiver adapted to receive the signal and to transmit the information, and a controller. The controller is coupled to the sensor(s), the power source and the transceiver, and is adapted to periodically cycle the module between a high power mode and a low power mode. During the high power mode, the information is received from at least one of the sensor(s) and transmitted. The period is associated with the time of activity instructions.

### FIELD

The present invention relates to monitoring devices.

### BACKGROUND

Soil monitors are commonly used to measure soil characteristics, such as pH or moisture content. The information from such monitors can be used to better allocate resources for soil care. For example, monitors may be used for measuring soil moisture levels in order to optimize irrigation schedules.

The power requirements for such monitors or monitoring systems can be substantial, and may require frequent battery replacement. An alternative to battery use is to hard-wire a power source to the monitoring system. However, such systems are often installed on farmland or other areas where animals and/or equipment traverse, either of which may cause damage to wires running along or beneath the surface.

### SUMMARY OF THE INVENTION

Forming one aspect of the invention is a module for use with a signal representative of time of activity instructions. The module comprises a power source in the form of one or more batteries, one or more sensors for receiving information, a transceiver adapted to receive the signal and to transmit the information, and a controller. The controller is coupled to the sensor(s), the power source and the transceiver, and is adapted to periodically cycle the module between a high power mode and a low power mode. During the high power mode, the information is received from at least one of the sensor(s) and transmitted. The period is associated with the time of activity instructions.

Further aspects of the invention will become apparent from the following description taken together with the accompanying drawings, the latter being briefly described hereinafter.

### BRIEF DESCRIPTION OF THE DRAWINGS

FIG. 1 is a schematic diagram of a module constructed in accordance with an embodiment of the present invention.

FIG. 2 is a schematic diagram of an alternate embodiment of the module of FIG. 1.

FIG. 3 is a schematic diagram of a base station constructed in accordance with an embodiment of the present invention.

FIG. 4 is a schematic diagram of a soil monitoring system constructed in accordance with an embodiment of the present invention.

### DETAILED DESCRIPTION

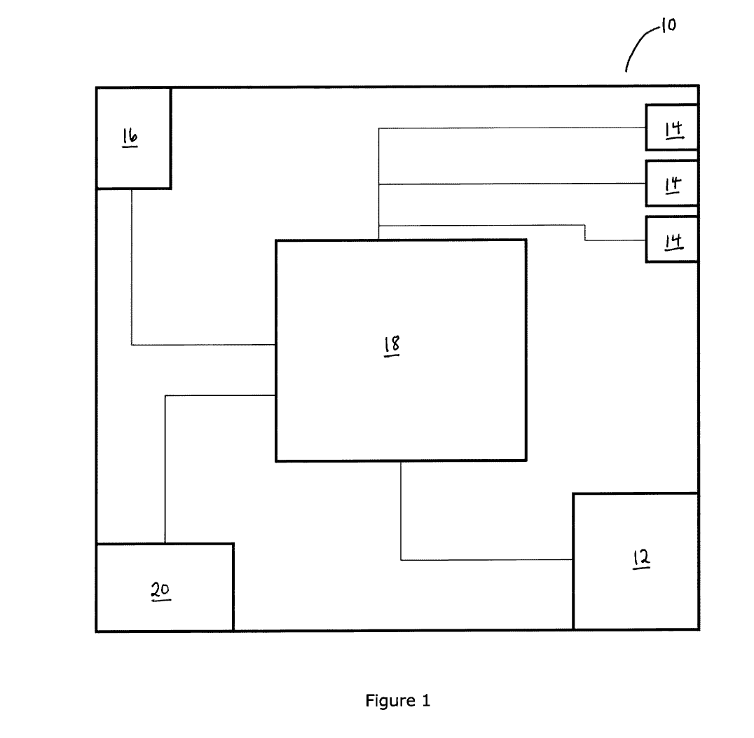

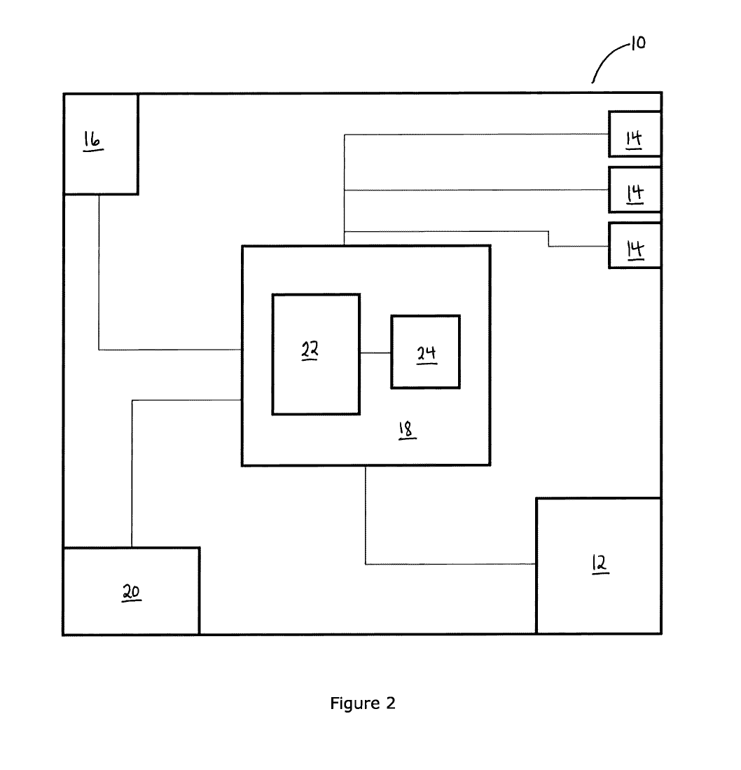

Referring to FIGS. 1 and 2, a module constructed in accordance with an embodiment of the invention is noted generally by reference numeral 10.

In the exemplary embodiment, module 10 includes a power source 12, one or more sensors 14 for receiving measured data or information, a wireless transceiver 16, and a controller 18 which includes a processor, and which may comprise a mini-computer or microcontroller. The controller is coupled to the sensor(s) 14, power source 12, and the transceiver 16. Module 10 may further include memory 20, which may be in the form of volatile memory or non-volatile flash memory.

Module 10 is adapted to receive a signal representative of time of activity instructions, and to transmit the sensor reading(s) or information. Controller 18 is configured to cycle module 10 periodically between a high power mode and a low power mode. During the high power mode, module 10 enters an active state during which it receives the sensor information from at least one of sensor(s) 14, and transmits the received sensor information, using transceiver 16, for storage and/or processing, typically at a remote location where the information can be accessed by a user.

The sensor information may be stored in memory 20 before its transmittal. Memory 20 will be sufficiently sized to store sensor data and configuration information that informs operation of module 10, such as the time of activity instructions. Any sensor data stored in memory 20 may be overwritten during each high power mode of module 10, when new sensor information is received and written to memory 20. Alternatively, controller 18 may be configured to delete the sensor information from memory 20 once it has been transmitted, before module 10 enters the low power mode.

The period at which controller 18 cycles module 10 from the low power mode to the high power mode is represented by a time interval value contained in the time of activity instructions. At each expiry of the time interval, module 10 enters the high power mode to begin receiving the sensor readings and to transmit that information onward. The module operates in the high power mode for an active time period that is a subset of the time interval. Upon completion of the high power mode, module 10 enters the low power mode and operates in the low power mode for an inactive time that is the remainder of the time interval. As such, each time interval is comprised of the active time and the inactive time.

Referring to FIG. 2, controller 18 of module 10 may include first processor 22 and second processor 24 in coupled relation. In this embodiment, second processor 24 may be a low power processor having lower power requirements than first processor 22, and remains in operation for the duration of the time interval. First processor 22 remains inactive during the time interval except when module 10 is in the high power mode, during the active time. At each expiry of the time interval, second processor 24 sends a wake instruction or signal to first processor 22 to thereby wake the first processor 22. Upon waking, module 10 enters the high power mode during which it receives and transmits the sensor information during the active time through the use of first processor 24.

Each of the first and second processors 22, 24 may further include a mini-computer or microcontroller which includes the processor. Examples of low-power consumption processors, microcontrollers and/or mini-computers that may be used include those manufactured by Arduino™. and that sold under the trade-mark Raspberry Pi™ by the Raspberry Pi Foundation.

In the exemplary embodiment, power source 12 is in the form of one or more batteries. In order to facilitate conservation of battery power, the active time during which module 10 operates in the high power mode will typically be less than the inactive time during which module 10 operates in the low power mode, and the time interval may be configured such that the active time is significantly less than the inactive time. For example, the time interval may be configured to be four hours, while the active time (i.e. the time required to receive and transmit the sensor information) may be in the range of 10 ms to 1 second, for example. The low power second processor 24 in combination with the ability to set the time interval to a value much greater than the active time is expected to result in increased battery life, making a battery-based monitor and monitoring system more viable.

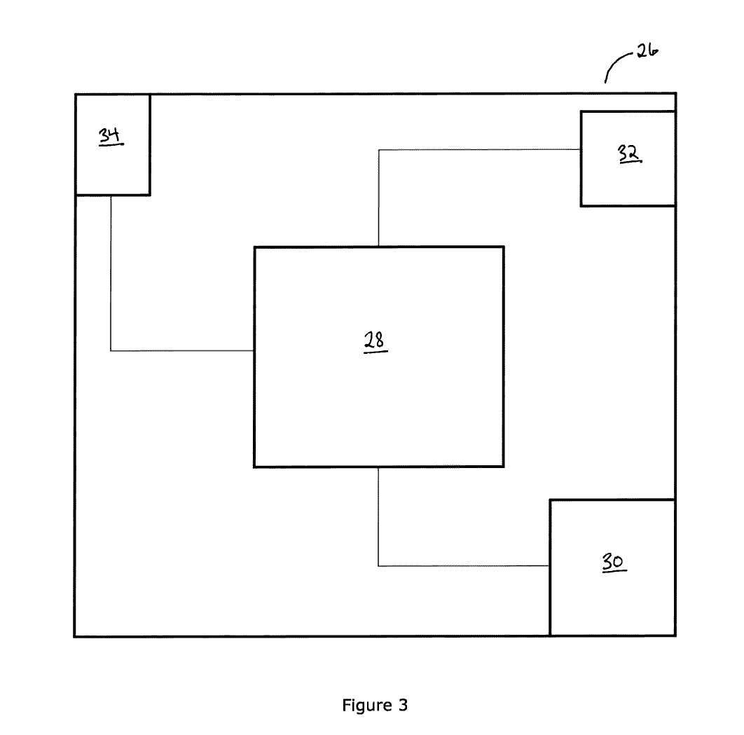

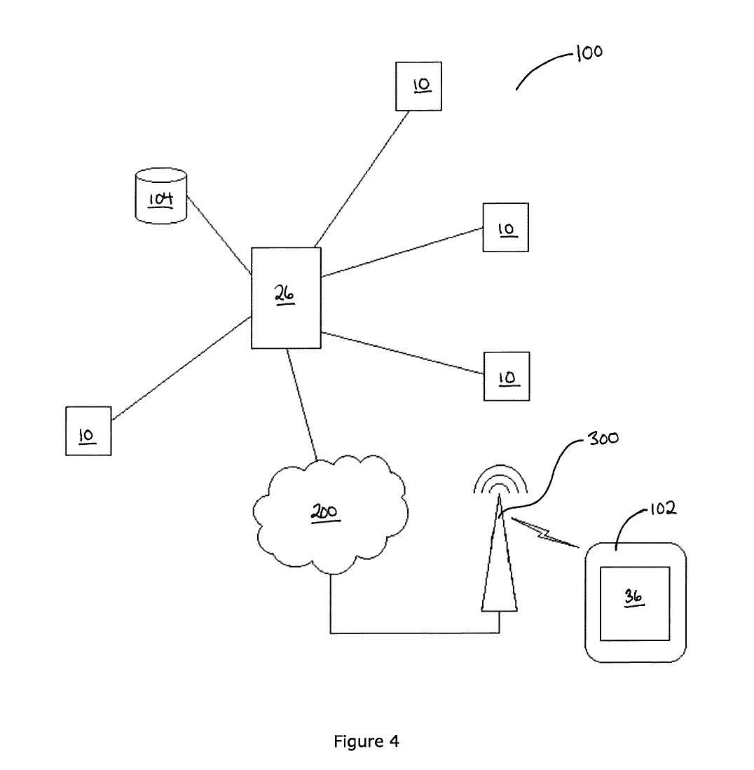

With reference to FIGS. 3 and 4, and in accordance with an alternate embodiment, there is provided a system 100. System 100 includes one or more of the modules 10, and a base station 26. Base station 26 includes a base station controller 28, a base station power source 30, a base station memory 32, and a wireless base station transceiver 34. Memory 32 may be in the form of volatile memory or non-volatile flash memory. Controller 28 is coupled to power source 30, memory 32 and transceiver 34, and is configured to produce the signal representative of time of activity instructions, and to receive, through transceiver 34, the sensor information transmitted by one or more of the module(s) 10.

Base station 26 and module(s) 10 communicate wirelessly through the use of wireless transceivers 34 and 16, respectively. Modules 10 can communicate directly with base station 26 or may communicate via other modules. Further, base station 26 will often be communicatively coupled to network 200, which may be a private network or a public network.

Typically, base station 26 will wirelessly communicate with network 200 through the use of transceiver 34. The communication can be established through mesh network, or star network or other methods (such as repeaters etc.) to make sure modules can transmit data to the Base Station or other modules over an infinite area.

Referring to FIG. 4, system 100 may further include a base station user interface 36. User interface 36 may be physically integrated with base station 26 and accessible through an interactive panel or display on the base station (not shown). Typically, user interface 36 will be a graphical user interface (GUI) remotely accessible over network 200 by a user on a device 102. Device 102 may be, for example, a desktop computer, smart phone, tablet, or any other digital device capable of displaying the GUI to the user and of receiving input from the user for interactions with the GUI (e.g., by touch on a touch-screen, mouse input, keyboard input, voice command, etc.).

Network 200 may include a combination of different types of interconnected networks, such as a private corporate or personal network, the Internet, a cellular network such as an LTE network, and a WiMax network, for example. FIG. 4 depicts device 102 as a wireless communications device capable of accessing network 200 via base transceiver station (BTS) 300. BTS 300 connects wireless device 102 to network 200, which will in such cases include a cellular network such as a UMTS, LTE, or GSM network, for routing data between the base station 26 and the device 102.

System 100 may further include a database 104. Database 104 may be locally stored within base station 26, or located remotely from base station 26 as shown in FIG. 4. Database 104 may include a database management system (DBMS) such as a relational database management system (RDBMS). Further, database 104 may comprise a distributed server architecture including multiple databases capable of synchronizing their content for redundant data storage.

Base station 26, upon receiving the sensor information from the module(s) 10, may store the information in database 104 and/or memory 32. Typically, where database 104 resides on a separate server, as shown in FIG. 4, the server will have greater memory storage capacity than that of base station 26 to allow for the storage of a history of sensor readings. The RDBMS will permit a user, using user interface 36, to access the records of database 104 via the base station controller 28 to thereby query the database and retrieve useful information, such as the most recently received sensor data, a history of soil moisture content over the past 12 months, etc.

Each module may be associated with a unique identifier. The modules may be associated with their respective identifiers within database 104 so that base station 26 is able to associate the received sensor information with the module from which it originates. Each module 10 may further include a global positioning system (GPS) and may additionally transmit its geographical coordinates to the base station 26. The coordinates may be transmitted with each transmission of the sensor information, or only when the module is first powered on, for example.

Base station 26 may be further configured to transmit to module(s) 10, through wireless transceiver 34, configuration information which may govern some or all of the operation of the module(s). The configuration information may be configurable by a user through the user interface 36, and may comprise one or more configuration settings stored in database 104 and/or memory 32. The configuration information may be transmitted to the module(s) 10 by activation of a transmission function by a user through the user interface 36, and/or base station 26 may be configured to transmit configuration information to the module(s) at a regular interval which may be preset by the user using user interface 36.

The configuration information may include, for example, identifier information identifying one or more of the module(s) 10 to be updated by the configuration information. As such, where, for example, five modules having identifiers 1A, 2A, 3A, 4A, and 5A have been deployed as part of system 100, and the user has updated the identifier information to include modules 1A and 4A only, then either: (i) base station 26 transmits the configuration information only to modules 1A and 4A on the next such transmission; or (ii) base station 26 broadcasts a message to all module(s) 10 and only modules 1A and 4A apply the configuration information.

The configuration information may also include monitoring information identifying information of interest (e.g., pH and moisture content only) to be monitored. The configuration information may additionally include the time of activity instructions and thus the time interval, the value of which may also be configurable by the user through user interface 36. Further, a user may be able to customize the configuration settings (e.g., the time interval, the information of interest, etc.) on a module-specific basis. Each module 10 may store the configuration information in memory 20. Similarly, the configuration may vary with calendar date, such that, for example, soil temperature readings end at the first day of winter and commence on the first day of spring, to coincide with periods relevant to crop yield.

For the purpose of illustrating an example use of system 100, one or more modules 10 may be utilized as soil monitors and inserted into soil at various distances from base station 26 on farmland, for example. Each module and may include sensors 14 for measuring soil temperature, pH, moisture content, CO₂ levels, and O₂ levels. Module(s) 10 may be completely buried within the soil. Once each module 10 is powered on (which may occur, for example, once power is supplied to the module, or through the use of a switch (not shown)), each module 10 may transmit to base station 26 an initial contact message indicating that it is ready to begin monitoring for, and transmitting, sensor data. The module(s) may optionally transmit GPS coordinates at this time. Base station 26 may transmit to the module(s), in reply to the initial contact message, the configuration information, or simply the time of activity instructions. Alternatively, base station 26 may not transmit any configuration information or instructions until directed to do so by a user through interaction with user interface 36. Once the configuration information or time of activity instructions is received by the module(s), the module(s) are accordingly updated and may begin to operate in accordance with the received configuration settings.

For example, and with reference to the previously discussed modules 1A-5A, the time interval may have been set to 6 hours for all modules. Modules 1A, 2A may have been configured to measure pH and moisture content only, while modules 3A, 4A and 5A may have been configured to additionally monitor CO₂ levels. For each time interval, each module operates in the low power mode until expiry of the time interval, at which point it transitions to the high power mode. During the active time that represents the duration of the high power mode, all modules will measure the soil pH and moisture content, while modules 3A-5A will additionally measure the CO₂ levels in their respective locations. The sensor information received is then transmitted by each module, optionally with GPS or other geographical coordinates, to base station 26 before each module re-enters the low power mode.

Base station 26 stores the received information in database 104 and/or memory 32 for later retrieval and/or analysis. Base station 26 may be further configurable to transmit alerts to device 102. The alert functionality and the threshold(s) for triggering an alert may be configurable by a user through user interface 36. For example, a user may configure base station 26 to send it an alert each time sensor information is received indicating that the soil moisture content corresponding to module 3A is below 20%. Module 3A may be located, for example, in an area where moisture loss is known to take place more quickly than elsewhere on the farmland. Receipt of such an alert by a user would provide useful information to help inform key decisions for soil care. For example, upon receipt of the alert, the user may activate the irrigation system in that area to hydrate the soil to acceptable levels. Alternatively, the irrigation system may automatically be activated to water plants based on pre-set parameters. Alerts may also be generated based upon, for example, battery status, which can be ascertained from a reading of battery voltage and comparison with hardware reference voltage.

Where user interface 36 runs as a background process on device 102, device 102 may trigger a visual and/or audible alert for the user once the alert is generated. Alternatively, or additionally, controller 28 of base station 26 may be configured to generate and transmit an email alert and/or text message alert based on contact information configured by the user through user interface 36.

The ability of each module 10 to operate in its low power mode for the bulk of its operation may facilitate the conservation of power. This is expected to be particularly beneficial in cases where modules 10 employ batteries as the power source 12. System 100 is thus expected to provide a customizable monitoring solution with remote access functionality and low power consumption properties for increased energy efficiency and an increased operating life where batteries are utilized. Users would be able to increase the duration of the low power mode, as desired, in order to lower the ratio of active: inactive time and thus increase the operating life, and thus the viability and practicality of, the battery-operated module(s) through online network (ex. Phone, computer, website).

It will be appreciated by the person of skill in the art that power source 12 may include an electrical infrastructure or grid rather than, or in addition to, batteries. Further, communication between the various system components and/or network 200 may take place over wired or partially wired connections or infrastructures.

Transceivers 16 and/or 34, as shown in the attached drawings, may instead consist of separate transmitters and receivers. Further, it will be appreciated that while module 10 and system 100 have been described herein with reference to soil monitoring, module 10 may be used to measure a variety of different types of data in a variety of different contexts. For example, one or more modules 10 may be placed at various locations (e.g., buildings, trees, etc.) in order to measure data such as atmospheric pressure, air moisture levels, and UV radiation levels. Further, the modules may be configured with high-power transceivers in order to transmit information over larger distances. In such cases, module 10 may be located in more remote areas, such as on a mountain-side, and may be configured to transmit collected information based on the available network facilitates (e.g., via satellite). The person of skill will appreciate that the number and/or types of sensors may vary. The skilled person will further appreciate that the GUI may be configured in a variety of formats (e.g., drop-down menus, radio buttons, text entry fields, etc., and any combination thereof), and may contain further configurable fields other than those transmitted to the module(s) as part of the configuration information, such as the alert contact information fields discussed above. Further, persons of ordinary skill will readily appreciate that the GUI may be functionalized such that it adjusts automatically to the addition or deletion of a module or sensor. In the exemplary embodiment, all of the modules are in the same channel (frequency) and have the same protocols and the GUI will make adjustments based upon the addition of new signals sharing these characteristics, but it will be appreciated that this is not strictly necessary

It is to be understood that what has been described are specific embodiments of the invention, and the invention is not so limited.

For example, whereas the exemplary module comprises a power source in the form of one or more batteries, the module could include a solar panel or more.

Accordingly, the scope of the claims should not be limited by the embodiments set forth above, but should be given the broadest interpretation consistent with the description as a whole.

1. A module for use with a signal representative of time of activity instructions, the module comprising:
a power source;
one or more sensors for receiving information;
a transceiver adapted to receive the signal and to transmit the information;
a controller coupled to the sensor(s), the power source and the transceiver, and adapted to periodically cycle the module between
a high power mode wherein the information is received from at least one of the sensor(s) and transmitted; and
a low power mode,
the period being associated with the time of activity instructions.

2. The module of claim 1 wherein the time of activity instructions includes a time interval representing said period.

3. The module of claim 2 wherein the module enters said high power mode at each expiry of said time interval and operates in said high power mode for an active time of said time interval.

4. The module of claim 3 wherein said module enters said low power mode at completion of said high power mode and operates in said low power mode for an inactive time that is a remainder of said time interval.

5. The module of claim 4 wherein said active time is less than said inactive time.

6. The module of claim 4 wherein said controller includes first and second processors coupled to each other.

7. The module of claim 6 wherein said second processor is a low power processor having lower power requirements than said first processor.

8. The module of claim 7 wherein said second processor remains in operation during said time interval and said first processor remains inactive during said time interval except during said active time.

9. The module of claim 8 wherein said second processor wakes said first processor at said each expiry of said time interval for the module to enter said high power mode to receive and transmit said information during said active time.

10. A system comprising:
one or more of the modules of claim 1; and
a base station adapted to produce the signal and adapted to receive the transmitted information.

11. The system of claim 10 wherein said base station includes a wireless transceiver and communicates with said module(s) wirelessly.

12. The system of claim 10 wherein said base station is communicatively coupled to a network.

13. The system of claim 12 wherein said communicative coupling between said base station and said network is wireless.

14. The system of claim 12 wherein said network is the Internet.

15. The system of claim 12 further comprising a user interface for said base station accessible by a user over said network.

16. The system of claim 15 wherein said base station is adapted to transmit to said module(s) configuration information, said configuration information configurable by the user through said user interface.

17. The system of claim 16 wherein said configuration information includes identifier information identifying one or more of said module(s) to be updated by said configuration information.

18. The system of claim 16 wherein said configuration information includes monitoring information identifying information of interest to be monitored by said module sensor(s).

19. The system of claim 16 wherein said configuration information includes said time of activity instructions, the time of activity instructions including a time interval at each expiry of which the module(s) enter said high power mode.

20. The system of claim 19 wherein a value of said time interval is configurable by said user interface.

21. A module according to claim 1, wherein the sensors combine GPS and LPS functionality.

22. A module according to claim 1, wherein the power source is defined at least in part by one or more of a battery and a solar panel.

23. A system according to claim 1, wherein each sensor has a unique proprietary identifier and, in use, the base station uses the identifier to identify the sensor and adjust the values sensed and transmitted accordingly.
